(19) United States
(12) Patent Application Publication
(10) Pub. No.: US 2012/0128609 A1
(43) Pub. Date: May 24, 2012

(54) COSMETIC USE OF NATURAL EMULSIFIERS

(76) Inventors: Sergio Amari, Paderno d'Adda (IT); Alain Thibodeau, Quebec (CA)

(21) Appl. No.: 13/126,906

(22) PCT Filed: Nov. 13, 2008
(86) PCT No.: PCT/IT08/00710

§ 371 Date: Jun. 23, 2011

(51) Int. Cl. A61K 8/92; A61Q 17/04; A61K 8/97

(52) U.S. Cl. 424/60; 424/59

(57) ABSTRACT

The use of natural emulsifiers for the preparation of cosmetics comprising a wax, an active ingredient and an emulsifier which allows the formation of an oil-in-water emulsion is disclosed. Preferably, said natural emulsifier is olive oil transesterified with cetearyl alcohol and/or sorbitol.

The present invention refers to the cosmetic use of natural emulsifiers, in particular for an improved action of the products for skin protection from sun rays.

By now it is widely known that sunshine has some harmful effects on human skin, which may end up causing even rather serious dermatological conditions. Acute rash and photoageing in are particularly well-known.

As regards rash, it generally appears after about twenty minutes of exposure to sunshine.

Instead, as photoageing is concerned instead, it has early and extended skin ageing as a consequence, as can be easily verified on the face of people used to spending a large proportion of their time in the sunshine, such as sailors and ski instructors.

Among the main causes of these effects, light radiation having wavelengths ranging between 290 and 320 nm are found, the so-called ultraviolet B rays (UVB) and those ranging between 320 and 400 nm, the so-called ultraviolet A rays (UVA). UVB radiation has a greater energy content and is the main causes of burns, while UVA radiation is more penetrating and leads to photo-ageing, even of the deepest skin layers. All ultraviolet radiation, both A and B, promotes notoriously very dangerous skin cancers.

Generally, in order to protect the skin against these problems, it has been advised—already for a long time—to use cosmetic products for sun protection, such as sun lotions and oils, which contain suitable substances, capable of screening sun rays.

It has already been seen in the past that the emulsifiers employed in cosmetics are not neutral, but in general affect the performances of cosmetic creams.

On this basis the present invention has been devised, consisting in the cosmetic use of natural emulsifiers for the preparation of cosmetics, comprising a wax, an active ingredient and an emulsifier, characterised in that the emulsifier allows the formation of an oil-in-water emulsion.

The present invention will now be described in greater detail. By natural emulsifiers, in the present description and in the attached claims emulsifiers are intended obtained by using mainly natural raw materials, leaving to synthetic substances the role of catalysts or, in any case, a marginal role. Various types of natural emulsifiers exist, of various origins. Among these, the present invention concerns the use of the ones capable of forming oil-in-water mixtures.

A particularly suited composition to form such mixtures is a transesterified olive oil. In particular, olive oil transesterified with cetearyl alcohol and/or sorbitol is useful.

In order to obtain the emulsifier to be used according to the present invention, it is possible to operate according to the methods known in organic chemistry for transesterification. For example, it is possible to mix with a vigorous shaking olive oil with the desired alcohol or alcohols at a temperature ranging between 20 and 100° C., in the presence of acidic catalysts, such as hydrochloric acid, sulphuric acid, nitric acid, BF₃, AlCl₃, trifluoroacetic acid, or basic catalysts, such as washing soda, potash, ammonia, sodium acetate. Another possibility is to saponify olive oil and to esterify the obtained said and/or acid derivatives with the alcohols. According to the methods known in the cosmetic industry, the emulsifier is added to an ordinary cosmetic product in an amount ranging between 3 and 50% by weight, preferably between 10 and 40% by weight, most preferably between 15 and 25% by weight. In the case of products for the protection from sun rays, such addition causes the effect of an amplified protection, with a protection effect increased by between 20 and 50%.

In the following, the present invention is further illustrated through operative examples, concerning the use in sun creams.

### EXAMPLE 1

The SPF parameters (according to Diffey's equation), star rating (UVA/UVB and λ_{c} ratio) and photostability of a series of sunscreens comprising ethyl-exyl methoxycinnamate (OMC), octocrylene, ethyl exyl/diethyl amino hydroxybenzoyl hexylbenzoate (DHHB/OMC), diethyl amino hydroxybenzoyl hexylbenzoate (DHHB), butyl methoxy dibenzoylmethane (BMDBM), silica/titanium dioxide have been checked.

The tests have been carried out on samples as such and on samples emulsified with olive oil transesterified with cetearyl alcohol and/or sorbitol. An SPF increase by 22% has been detected for the pair BMDBM/OMC, by 32% for the pair DHHB/OMC. The other parameters have also improved.

### EXAMPLE 2

The samples with olive oil transesterified with cetearyl alcohol and/or sorbitol as emulsifier have been tested with a series of emollients. A marked improvement of all the properties has taken place with C₁₂₋₁₅ alkyl benzoate.

1-10. (canceled)

11. A cosmetic, comprising a wax, an active ingredient, and a natural emulsifier, wherein the natural emulsifier provides the formation of an oil-in-water emulsion.

12. The cosmetic of claim 11, wherein the natural emulsifier is transesterified olive oil.

13. The cosmetic claim 12, wherein the olive oil is transesterified with cetearyl alcohol and/or sorbitol.

14. The cosmetic of claim 11, further comprising an emollient, wherein the emollient is C12-15 alkyl benzoate.

15. The cosmetic of claim 11, wherein the natural emulsifier is present in an amount ranging between 3 and 50% by weight of the cosmetic.

16. The cosmetic of claim 11, wherein the natural emulsifier is present in an amount ranging between 10 and 40% by weight of the cosmetic.

17. The cosmetic of claim 11, wherein the natural emulsifier is present in an amount ranging between 15 and 25% by weight of the cosmetic.

18. The cosmetic of claim 11, wherein the active ingredient is a sunscreen that provides protection from sun rays.

19. The cosmetic of claim 18, wherein the sunscreen of is a mixture of butyl methoxy dibenzoylmethane and ethyl exyl methoxycinnamate.

20. The cosmetic of claim 18, wherein the sunscreen is a mixture of diethyl amino hydroxybenzoyl hexylbenzoate and ethyl exyl methoxycinnamate.
